(19) United States
(12) Patent Application Publication
(10) Pub. No.: US 2026/0020988 A1
(43) Pub. Date: Jan. 22, 2026

(54) TACTICAL BANDAGE PRESSURE BAR

(71) Applicant: ALPHAPOINTE, Kansas City, MO (US)

(72) Inventors: CHRISTOPHER BRYAN KOSIOREK, LA Vernia, TX (US); Ryan WILLIAMS, Olathe, KS (US); Cooper BREDEHOEFT, Kansas City, KS (US)

(21) Appl. No.: 19/272,600

(22) Filed: Jul. 17, 2025

Related U.S. Application Data
(60) Provisional application No. 63/673,107, filed on Jul. 18, 2024.

(51) Int. Cl. A61F 13/05; A61F 13/00

(52) U.S. Cl. CPC A61F 13/05; A61F 2013/0028; A61F 2013/00468

(57) ABSTRACT

A pressure bar for applying pressure to a wound is provided. The pressure bar includes a pressure member defining a pressure application surface for facilitating application of a generally even distributed pressure to a wound. The pressure member includes a clip for quickly and easily engaging and disengaging the pressure bar with a bandage, thereby facilitating quick replacement of bandages, when necessary. Cross members of the pressure bar are configured to engage with a tourniquet or other pressure application device, thereby facilitating prolonged application of pressure to the wound.

### CROSS-REFERENCE TO RELATED APPLICATIONS

This application claims priority pursuant to 35 U.S.C. 119 (e) to co-pending U.S. Provisional Patent Application Ser. No. 63/673,107, filed Jul. 18, 2024, the entire disclosure of which is incorporated herein by reference.

### FIELD OF THE INVENTION

The present invention relates generally to medical devices. More specifically, the present invention is concerned with a small, lightweight, easy-to-use device for applying pressure to a wound.

### BACKGROUND

A large number of potentially survivable casualties result in mortality due to uncontrolled hemorrhage. Accordingly, it would be beneficial to have a readily accessible hemorrhage control device. Bandages and other wound treatment devices can help to stop or control hemorrhaging, but they require pressure to be applied to a wound. In many cases, the application of relatively consistent pressure to a wound is critical to increase the chances of survival. Accordingly, it would be beneficial to have a system for and method of applying relatively consistent localized pressure to a wound.

Many injuries happen away from a primary care facility, such as a hospital. Accordingly, initial triage is often required before transporting patients to the care facility. Depending on the distance, resource availability, number of casualties, and a number of other factors, patients may experience long delays between initial triage and receiving final care, during which time it may be necessary to apply relatively constant pressure to a wound. Accordingly, it would be beneficial to have a system for and a method of utilizing a tourniquet or other pressure application device to apply relatively consistent pressure to a wound for an extended period of time.

In some embodiments, it may be necessary or advisable to relieve pressure at one location and apply pressure to another location, with the two locations possibly being associated with different patients. Accordingly, it would be beneficial to have a pressure application device that can be quickly and easily removed from a first location, potentially while leaving an associated first bandage in place, and redeployed at a second location with a second bandage.

### SUMMARY

The present invention comprises a readily accessible hemorrhage control device. In some embodiments, the present invention comprises a pressure bar that is configured to apply relatively consistent localized pressure to a wound. In some embodiments, the pressure bar is configured to be used with a tourniquet, a belt, or the like (each a “tourniquet”), thereby facilitating application of relatively consistent pressure to a wound for an extended period of time. In some such embodiments, the pressure bar includes a pressure member extending between two cross members, each cross member having opposed flanges for defining a channel for engaging with a tourniquet.

The present invention is configured to quickly and easily engage with, and disengage from, a bandage, thereby facilitating selective deployment and redeployment, as necessary. In some embodiments, the pressure member includes a clip for engaging with a loop of a bandage.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION OF THE DRAWINGS

A preferred embodiment of the invention, illustrative of the best mode in which the applicant has contemplated applying the principles, is set forth in the following description and is shown in the drawings and is particularly and distinctly pointed out and set forth in the appended claims.

FIG. 1 is a perspective view of a pressure bar of the present invention.

FIG. 2 is a top view of the pressure bar of FIG. 1.

FIG. 3 is a side view of the pressure bar of FIG. 1.

FIG. 4 is the side view of FIG. 3 shown with a tourniquet engaged therewith.

FIG. 5 is an end view of the pressure bar of FIG. 1.

FIG. 6 is the end view of FIG. 5 shown with a tourniquet engaged therewith.

FIG. 7 is a perspective view of a bandage of certain embodiments of the present invention.

FIG. 8 is a top view of a pressure bar of the present invention engaged with a bandage of the present invention.

### DETAILED DESCRIPTION (OPTIONAL)

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

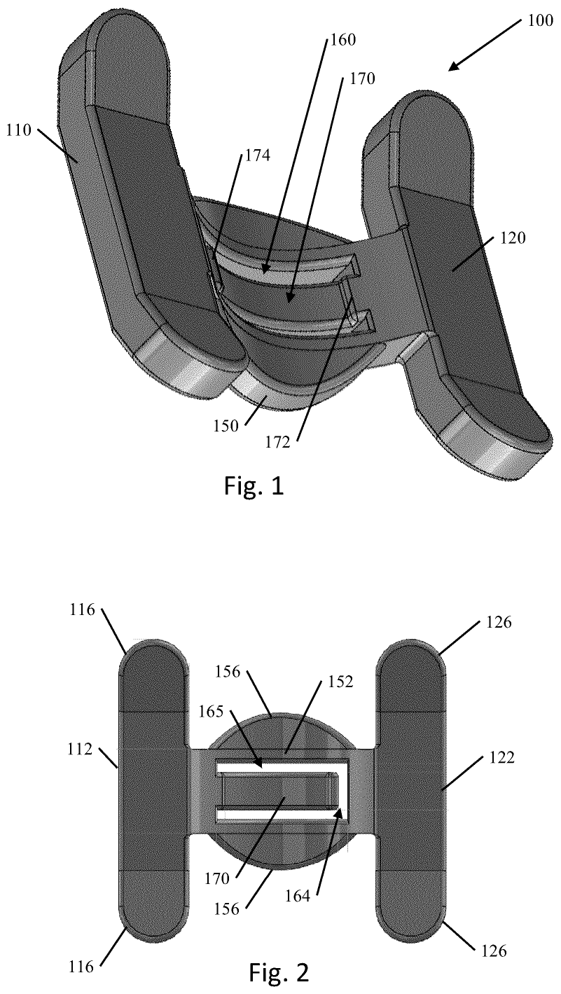

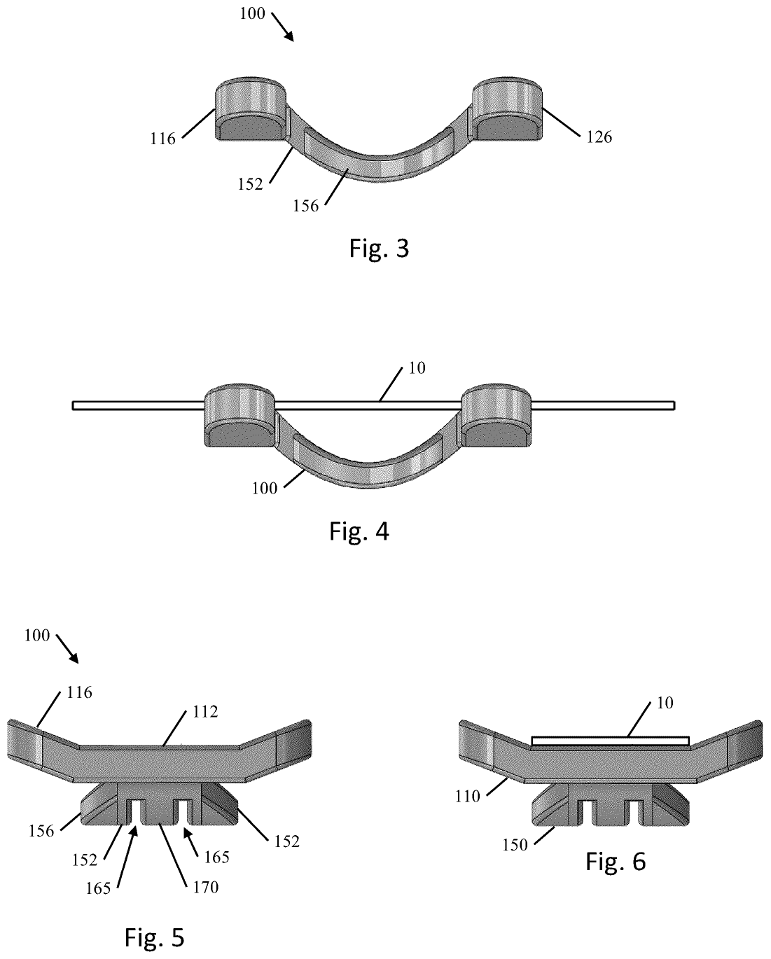

Referring to FIGS. 1 and 2, the present invention includes a pressure bar 100 having a pressure member 150 extending between opposed first 110 and second 120 cross members. In some embodiments, each cross member comprises a main section 112, 122 extending between opposed flanges 116, 126. In some such embodiments, the flanges define a channel or other area (a “channel”) for engaging with a tourniquet, such as shown in FIGS. 4 and 6. In some embodiments, each extends at a first angle relative to its respective main section and a second angle relative to its corresponding opposed flange. In some such embodiments, the first angle is between 20 and 25 degrees, and the second angle is between 140 and 130 degrees. In some such embodiments, the first angle is about 21 degrees, and the second angle is about 138 degrees.

Still referring to FIGS. 1 and 2, the pressure member 150 of the present invention comprises a main body 152 having opposed first and second ends coupled to respective first 110 and second 120 cross members, the pressure member being configured for applying pressure to a wound. In some embodiments, the pressure member 150 comprises opposed flanges 156 extending from respective sides of the main body 152, thereby increasing a surface area for applying pressure to the wound. In some such embodiments, the flanges define a curved outer edge, thereby defining a generally circular area for applying pressure to the wound.

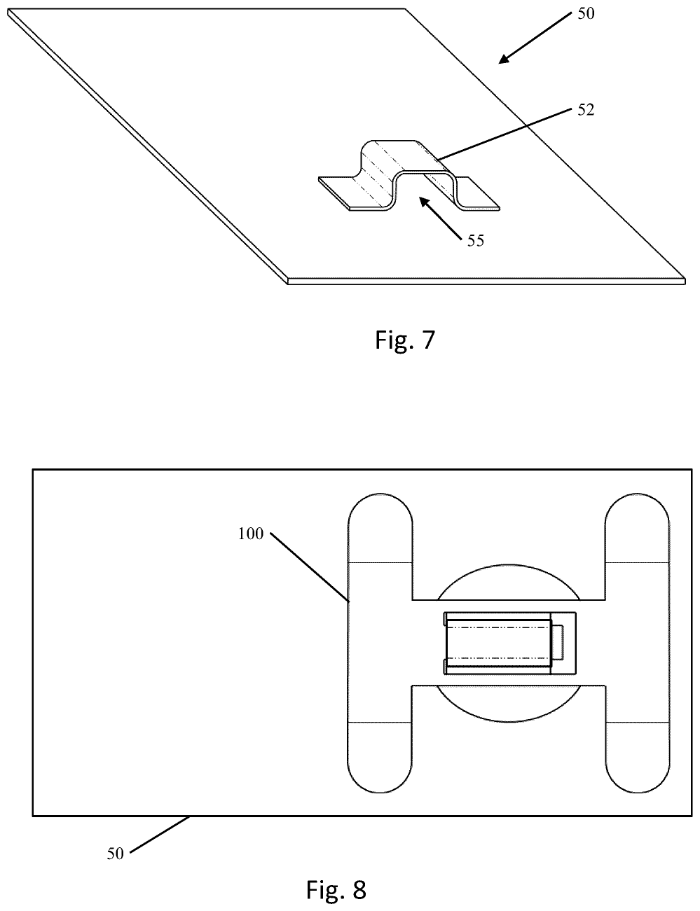

Referring to FIGS. 7 and 8, the pressure bar 100 of the present invention is configured to engage with a bandage 50, thereby facilitating pressing the bandage against a wound. In some embodiments, the bandage 50 includes a loop 52 that is configured to engage with the pressure bar 100, thereby limiting movement of the bandage 50 relative to the pressure bar 100. In some such embodiments, the pressure member 150 defines an aperture 160 and a clip 170 positioned therein. In some embodiments, the clip 170 includes opposed proximal 172 and distal 174 ends, the proximal end 172 being coupled to the main section 152 of the pressure member 150, and the distal end 174 being positioned in the aperture 160 such that the clip 170 is a cantilever member extending through a portion of the aperture 160, thereby defining an end gap 164 of the aperture 160 to facilitate moving the pressure bar 100 in and out of engagement with the loop 52 of the bandage 50. The position of the clip 170 within the aperture 160 further defines opposed side gaps 165 of the aperture 160, the side gaps being configured for receiving respective side portions of the loop 52 of the bandage 50 while the pressure bar 100 is engaged with the bandage 50. In some embodiments, the distal end 174 of the clip 170 defines a lip for catching an edge of the loop 52, thereby inhibiting inadvertent disengagement of the bandage 50 from the pressure bar 100. In some such embodiments, the lip extends vertically upward from a top surface of the clip. In some embodiments, the proximal end 172 of the clip 170 defines a ledge. In some such embodiments, the top surface of the clip 170 defines a recessed area extending between the lip and the ledge, the recessed area being configured for receiving a top portion of the loop 52.

In some embodiments, the pressure member 150 extends vertically downward and away from the cross members, thereby facilitating direct pressure on a wound. In some such embodiments, a bottom surface of the pressure member 150 is a pressure application surface of the pressure member 150. In some embodiments, the pressure application surface is defined by a curved surface. In some such embodiments, the curved surface is a defined by a portion of a cylindrical shape.

In some embodiments, the present invention comprises utilizing a pressure bar of the present invention to apply pressure to a wound. In some such embodiments, the method comprises engaging the pressure bar 100 with a bandage 50 and utilizing the pressure bar to distribute pressure generally evenly over a pressure area defined by a pressure member 150 of the pressure bar 100. In some such embodiments, engaging the pressure bar 100 with the bandage 50 includes pulling a loop 52 of the bandage 50 through an end gap 164 and onto a clip 170 of the pressure bar 100 such that the clip 170 extends through a void 55 defined by the loop 52. In some embodiments, pulling the loop 52 onto the clip 170 includes pulling the loop over a lip defined by a distal end 174 of the clip 170. In some such embodiments, engaging the loop 50 with the pressure bar 100 includes nesting a top portion of the loop 52 in a recessed area of the clip 170 while side portions of the loop 52 extend around the clip 170 through side gaps 165 associated therewith.

In some embodiments, methods of the present invention include engaging a tourniquet 10 with the pressure bar 100 to facilitate application of a generally constant pressure for an extended period of time. In some such embodiments, the tourniquet engages with first 110 and second 120 cross members and extends over a pressure member 150 extending therebetween. In some embodiments, the tourniquet 10 is positioned between respective flanges 116, 126 of the first 110 and second 120 cross members, thereby inhibiting inadvertent disengagement of the tourniquet 10 from the pressure bar 100.

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the inventions is by way of example, and the scope of the inventions is not limited to the exact details shown or described.

Although the foregoing detailed description of the present invention has been described by reference to an exemplary embodiment, and the best mode contemplated for carrying out the present invention has been shown and described, it will be understood that certain changes, modification or variations may be made in embodying the above invention, and in the construction thereof, other than those specifically set forth herein, may be achieved by those skilled in the art without departing from the spirit and scope of the invention, and that such changes, modification or variations are to be considered as being within the overall scope of the present invention. Therefore, it is contemplated to cover the present invention and any and all changes, modifications, variations, or equivalents that fall with in the true spirit and scope of the underlying principles disclosed and claimed herein. Consequently, the scope of the present invention is intended to be limited only by the attached claims, all matter contained in the above description and shown in the accompanying drawings shall be interpreted as illustrative and not in a limiting sense.

Having now described the features, discoveries and principles of the invention, the manner in which the invention is constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the invention herein described, and all statements of the scope of the invention which, as a matter of language, might be said to fall therebetween.

What is claimed is:

1. A pressure bar for applying pressure to a wound, the pressure bar comprising:
opposed first and second cross members; and
a pressure member extending between the opposed first and second cross members,
wherein the pressure member comprises a clip for selectively engaging with a loop of a bandage.

2. The pressure bar of claim 1, wherein said pressure member further comprises a main section coupled to respective main sections of the first and second cross members, said main section of the pressure member defining an aperture within which the clip is positioned.

3. The pressure bar of claim 2, wherein said clip is a cantilever member having opposed proximal and distal ends, said proximal end of said clip being coupled to said main section of said pressure member, and said distal end of said clip being positioned in said aperture.

4. The pressure bar of claim 3, wherein said aperture comprises an end gap extending from said distal end of said clip, said end gap being configured to receive a top portion of the loop of the bandage while moving said clip in and out of engagement with the loop of the bandage.

5. The pressure bar of claim 4, wherein said aperture comprises opposed side gaps extending from said end gap, each side gap being configured to receive opposed side portions of the loop when said clip is engaged with the loop of the bandage.

6. The pressure bar of claim 5, wherein said clip defines a lip, a ledge, and a recessed area extending therebetween, said lip and said ledge being positioned at or near said distal and proximal ends, respectively, and said recessed area being configured to receive the top portion of the loop when said clip is engaged with the loop of the bandage.

7. The pressure bar of claim 6, wherein at least a portion of a bottom surface of said pressure member defines a pressure application surface, and wherein said pressure member extends vertically downward and away from said cross members such that said pressure application surface is nonplanar with said first and second cross members.

8. The pressure bar of claim 7, wherein said pressure application surface is defined by a portion of a cylindrical shape.

9. The pressure bar of claim 7, wherein said pressure member comprises opposed flanges, wherein a bottom surface of each flange defines a portion of the pressure application surface, and wherein each flange is displaced from the first and second cross members.

10. The pressure bar of claim 9, wherein said first and second cross members each comprise opposed flanges extending from opposed ends of respective main sections, each flange being angled relative to respective main sections such that said cross members define a channel for receiving a tourniquet.

11. A method of applying pressure to a localized area, the method comprising:
positioning a bandage over the localized area;
inserting a clip of a pressure bar into a loop of the bandage, thereby positioning a pressure member of the pressure bar against a top surface of the bandage, a pressure application surface of the pressure bar being defined by a bottom surface of the pressure member; and
applying pressure to opposed first and second cross members of the pressure bar, wherein the pressure member extends between the opposed first and second cross members such that applying pressure to the cross members results in pressure being applied by the pressure member to the localized area.

12. The method of claim 11, wherein inserting the clip into the loop comprises pulling a top portion of the loop through an end gap of the pressure member, thereby positioning opposed side portions of the loop in respective side gaps of the pressure member, the end gap and side gaps being formed by the clip extending into an aperture defined by the pressure member.

13. The method of claim 12, wherein inserting the clip into the loop further comprises:
pulling the top portion of the loop over a lip defined by a distal end of the clip; and
nesting a top portion of the loop in a recessed area of the clip, the recessed area extending between the lip and a ledge defined by a proximal end of the clip.

14. The method of claim 13, wherein the pressure member extends vertically downward and away from the first and second cross members such that the pressure application surface is nonplanar with the first and second cross members.

15. The method of claim 14, further comprising engaging a tourniquet with the first and second cross members, wherein applying the pressure to the cross members comprises constricting the tourniquet.

16. The method of claim 15, wherein the first and second cross members each comprise opposed flanges extending from opposed ends of respective main sections, each flange being angled relative to respective main sections such that the cross members define a channel for receiving the tourniquet, wherein the channel is configured to inhibit inadvertent disengagement of the tourniquet from the pressure bar while constricting the tourniquet.

17. The method of claim 11, further comprising positioning a tourniquet within a channel defined by the first and second cross members, wherein applying the pressure to the cross members comprises constricting the tourniquet.

18. A pressure system for applying pressure to a wound, the pressure system comprising:
a pressure bar having opposed first and second cross members and a pressure member extending therebetween, said pressure member defining a pressure application surface; and
a bandage comprising a loop defining a void,
wherein the pressure system is configured to move from an unclipped configuration to a clipped configuration by engaging a clip of said pressure member with said loop of said bandage.

19. The pressure system of claim 18, further comprising a tourniquet engaged with the first and second cross members, wherein said pressure member extends vertically downward and away from said cross members such that said pressure member is displaced from said tourniquet.

20. The pressure system of claim 19, wherein said clip defines a lip, a ledge, and a recessed area extending therebetween, wherein a top portion of said loop of said bandage nests within said recessed area of said clip when the pressure system is in the clipped configuration.
